(19) United States
(12) Patent Application Publication
(10) Pub. No.: US 2008/0021376 A1
(43) Pub. Date: Jan. 24, 2008

(54) Portable Mechanical Insulin Injection Device

(76) Inventor: Kyu-Dong Choi, Seoul (KR)

Correspondence Address: KENYON & KENYON LLP, 1500 K STREET N.W., SUITE 700, WASHINGTON, DC, 20005, US

(21) Appl. No.: 11/632,219

(22) PCT Filed: Mar. 7, 2006
(86) PCT No.: PCT/KR06/00776

§ 371 Date: Jan. 11, 2007

(30) Foreign Application Priority Data
Mar. 7, 2005 (KR) 10-2005-0018551

(51) Int. Cl. A61M 1/00; A61M 31/00

(52) U.S. Cl. 604/27; 604/93.01

(57) ABSTRACT

The present invention relates to a portable mechanical insulin injection device comprising i) a mechanical insulin injection device (10) wherein said injection device is mechanically operated by hand-operated rotating part (106) whereas the amount of injected insulin and the pre-determined injection amount of insulin are electrically controlled and displayed by electrical control panel (110); and E) an insulin injection unit (20) wherein an insulin injection needle (211) is inserted in the skin or muscle of patient and an insulin injection connector (215) is fixed with the insulin outlet of mechanical insulin injection device (10) at the time of insulin injection.

### TECHNICAL FIELD

The present invention relates to a portable mechanical insulin injection device. More specifically, it relates to a portable mechanical insulin injection device comprising a mechanical insulin injection device (10) and an insulin injection unit (20), wherein the insulin is mechanically injected by hand-operated rotating device and the amount of injected insulin and the pre-determined injection amount of insulin are controlled and displayed by electrical device in the said mechanical insulin injection device.

### BACKGROUND ART

Commercially marketed portable insulin injector has been used by fixing the insulin injection needle at the time of insulin injection, normally 3 or 4 times a day.

Since such portable insulin injector requires the fixing of the needle unit at the portable insulin injector whenever it is used, it has some inconvenience for injection. Further, it shall accompany the pains of the patient because needle unit shall be inserted into the skin or muscle of patient at the time of insulin injection. Therefore, it also induces the fear of insulin injection to the patient.

To overcome such handicaps, insulin pump has been developed for automatic injection of insulin. Even though it has an advantage for injecting the pre-determined injection amount of insulin at the pre-determined injection time interval, such insulin pump is so expensive that the patient requires high cost for using it.

On the other hand, an insulin pump may not be applied to the patient who has temporarily severed diabetes symptom, because this patient does not require continuous injection of insulin after a lapse of injection of insulin. Since it is not necessary for such kind of patient to purchase the high price insulin pump, low price portable insulin injection device has been required to replace the insulin pump.

Accordingly, the inventor of present invention disclosed portable insulin injection device in Korean Utility Model Registration No. 373,593. In this disclosure, a portable insulin injection device comprising cylinder unit, nut unit and plunger unit, wherein the rotation of plunger unit fixed with nut unit extrudes the insulin in the cylinder unit by pushing it. Further, such portable insulin injection device connects with insulin injection unit comprising needle, injection tube and connector to be fixed with cylinder unit. Moreover, the needle in insulin injection unit is inserted in the skin or muscle of patient.

The therapeutic insulin for treating diabetes can be classified into two types, such as, meal-associated (prandial) insulin and basal insulin. Meal-associated insulin is normally injected 3 times a day just before having a meal to prevent a rapid increase of blood sugar level, whereas basal insulin is injected once a day to maintain the insulin level constantly for 24 hours.

Intensive insulin therapy for diabetes in which insulin is injected 3 or 4 times a day can maintain the glycosylated hemoglobins (HbA1C) value within a normal range. Further, it has been reported that complicating disease raised from diabetes, such as, retinopathy or nephropathy can be protected in a certain degree by intensive insulin therapy. Even though complicating disease induced from diabetes occurs to the patient, such complicating disease can be controlled and relieved by intensive insulin therapy. The complicating disease, such as, neuropathy or blood vessel disease can be also reduced to a certain degree.

Therefore, intensive insulin therapy has been recommended to prevent the complicating disease induced from diabetes. According to DCCT report in 1993, the intensive insulin therapy treated group reduced the nephropathy about 34% compared to the conventional therapy treated group against diabetes. Further, in case of only 2% of patients treated by intensive insulin therapy, the albuminuria became worse into microalbuminuria. Of course, nephropathy also can be reduced. According to DCCT report in 1993, the intensive insulin therapy treated group reduced the retinopathy about 69% compared to the conventional therapy treated group against diabetes. Further, the level of glycosylated hemoglobins of diabetes patient treated by intensive insulin therapy is only 1% higher than that of normal group. As a conclusion, intensive insulin therapy is a primary treating method for preventing the complicating disease, such as, neuropathy.

According to DCCT report, in the intensive insulin therapy group, 43% of patients reduced the microalbuminuria as well as 56% of patients reduced the albuminuria as a result of 9 years observation. Further, occurrence of arteriosclerosis was also reduced according to the control of blood sugar. If the level of blood sugar is not controlled, the occurrence of arteriosclerosis or hyperinsulinemia may increase as to diabetes patients. Of course, at the group in which level of blood sugar is well controlled, hypercholesterolemia also can be reduced.

Therefore, the present invention developed a portable insulin injection device which is mechanically operated very precisely in order to replace the insulin pump. Further, the portable insulin injection device of the present invention can be afforded in a low price.

### DISCLOSURE OF INVENTION

The object of the present invention is to provide a portable mechanical insulin injection device comprising
i) a mechanical insulin injection device (10) wherein said injection device is mechanically operated by hand-operated rotating part (106) whereas the amount of injected insulin and the pre-determined injection amount of insulin are electrically controlled and displayed by electrical control panel (110); and
ii) an insulin injection unit (20) wherein an insulin injection needle (211) is inserted in the skin or muscle of patient and an insulin injection connector (215) is fixed with the insulin outlet of mechanical insulin injection device (10) at the time of insulin injection,

wherein said mechanical insulin injection device (10) comprises
a) an operating gear (105) which is mechanically operated by the rotation of hand-operated rotating device (106) through gear rotating part (108);
b) a plunger shifting screw (103) connected with operating gear (105), so that said plunger shifting screw (103) shifts the plunger gasket (102) by moving the plunger shift lock nut (104);
c) an insulin syringe (101) in which insulin is pushed out according to the shift of the plunger gasket (102); and
d) a housing having an opening for insulin outlet of mechanical insulin injection device.

Further, a portable mechanical insulin injection device further comprises an electrical device comprising
i) a control panel (110) controlling the insulin injection amount upon inputting the pre-determined injection amount by control button (113) and measuring the injection amount by sensor (107);
ii) a liquid crystal display (109) controlled by control panel (110);
iii) a warning sound speaker (111) generated according to the around injection of pre-determined insulin amount; and
iv) battery (112) as electric power source.

Further, said insulin syringe (101) in the insulin injection device (10) can be replaced by any type of insulin syringes such as meal-associated insulin syringe or with basal insulin syringe upon changing the tubulate extension part which shall be fixed at the insulin injection edge part (216).

Further, said insulin syringe (101) can be replaced by commercially marketed insulin cartridge.

### BRIEF DESCRIPTION OF THE DRAWINGS

FIG. 1 shows a total structure of the portable mechanical insulin injection device of the present invention.

FIG. 2 shows a mechanical part structure of the portable mechanical insulin injection device of the present invention.

FIG. 3 shows a electrical part structure of the portable mechanical insulin injection device of the present invention.

FIG. 4a shows a structure of insulin injection unit of the portable mechanical insulin injection device of the present invention.

FIG. 4b shows a replaceable part structure of insulin injection unit of the portable mechanical insulin injection device of the present invention.

*Description of Reference Numeral

| | |
|---|---|
| 10: | mechanical insulin injection device |
| 20: | insulin injection unit |
| 101: | insulin syringe |
| 102: | plunger gasket |
| 103: | plunger sshifting screw |
| 104: | plunger shift lock nut |
| 105: | operating gear |
| 106: | hand-operated rotating part |
| 107: | sensor |
| 108: | gear rotating part |
| 109: | liquid crystal display |
| 110: | control panel |
| 111: | warning sound speaker |
| 112: | battery |
| 113: | control button |
| 210: | non-woven fabric for skin adhesion |
| 211: | insulin injection needle |
| 212: | tubulate extension edge |
| 213: | tubulate extension part |
| 214: | tubulate extension part |
| 215: | insulin injection connector |
| 216: | insulin injection edge part |

### BEST MODE FOR CARRYING OUT THE INVENTION

The present invention can be explained more specifically according to drawings.

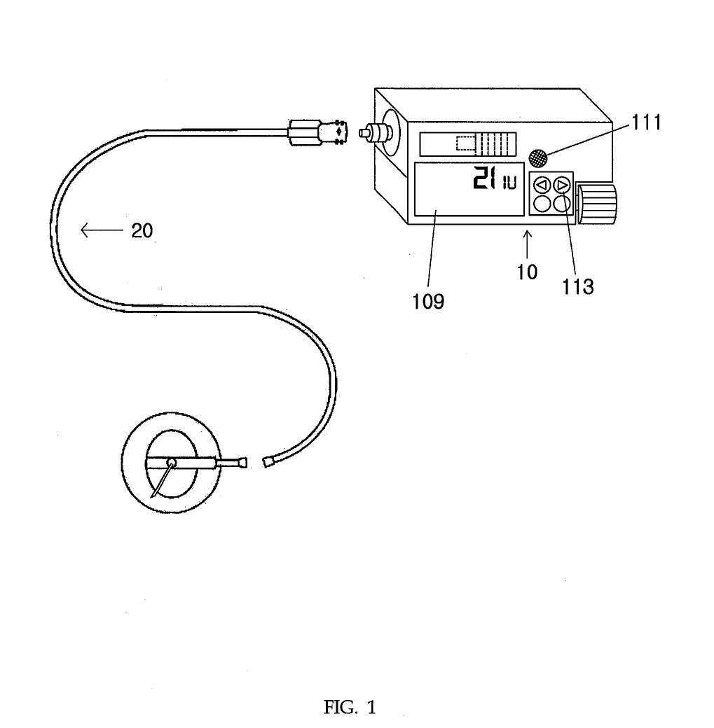

FIG. 1 shows a total structure of the portable mechanical insulin injection device of the present invention.

As shown in FIG. 1, the portable mechanical insulin injection device of the present invention comprises a mechanical insulin injection device (10) and an insulin injection unit (20). The portable mechanical insulin injection device of the present invention has been developed in order to inject the insulin very precisely by substituting the motor in the insulin pump operated by battery power source with the hand-operated rotating part. Therefore, the hand-operated rotating part of the present invention corresponds with the motor part of conventional insulin pump operated by battery power source. However, the portable mechanical insulin injection device of the present invention comprises a minimum electrical device for controlling and displaying the insulin injection amount.

In case of electrically operated insulin pump, the insulin injection part shall be mechanically mounted as well as insulin injection syringe shall be continuously replaced, even though insulin pump is controlled by electrical digital device. Further, the impact of insulin pump or user's mis-operation of insulin pump induces the damage of electrical digital part of insulin pump as well as mechanical part of insulin pump.

However, the portable mechanical insulin injection device of the present invention seldom takes any damages from outside impact or any user's mis-operation, since major power part of the present invention is mechanically operated. Further, the portable mechanical insulin injection device of the present invention can afford much lower price compared to conventional digital insulin pump since most part of device is mechanically operated. Of course, minimum electrical part, such as control panel, liquid crystal display and warning sound speaker requires battery power source.

On the other hand, the portable mechanical insulin injection device of the present invention can use any type of insulin injection, such as meal-associated insulin injection or basal insulin injection. Conventionally, only meal-associated insulin can be injected by insulin syringe, whereas basal insulin is injected by additional injection device. However, the replacement of insulin injection unit of the present invention enables the portable mechanical insulin injection device to inject both meal-associated insulin and basal insulin. Of course, the insulin injection unit shall be connected with insulin injection edge part (26).

Further, said insulin syringe in the portable mechanical insulin injection device of the present invention can be replaced by commercially marketed insulin cartridge since the mechanical insulin injection device (10) is designed in order to place the insulin cartridge.

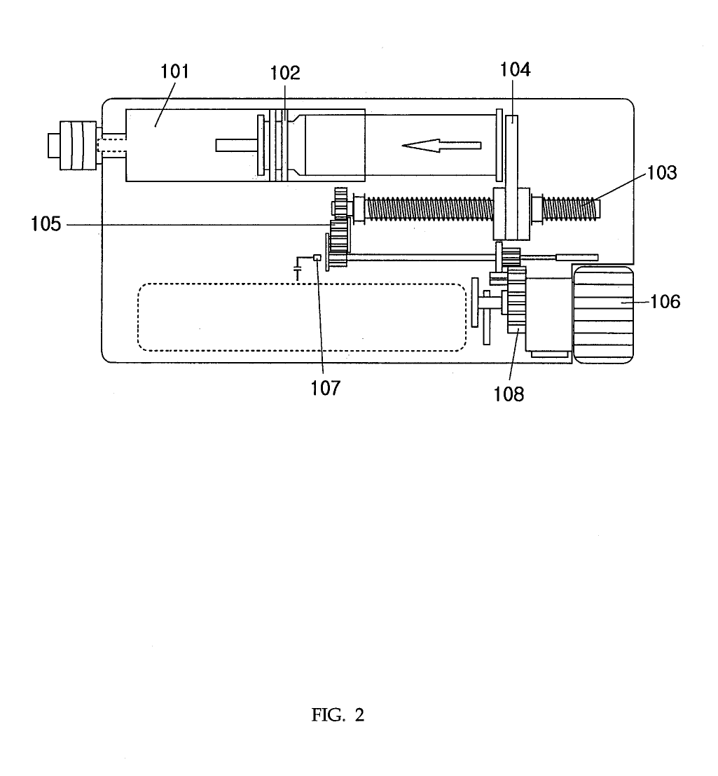

FIG. 2 shows a mechanical part structure of the portable mechanical insulin injection device of the present invention.

As shown in FIG. 2, said mechanical insulin injection device has following parts and functions; an operating gear (105) which is mechanically operated by the rotation of hand-operated rotating device (106) through gear rotating part (108); a plunger shifting screw (103) connected with operating gear (105) so that said plunger shifting screw (103) shifts the plunger gasket (102) by moving the plunger shift lock nut (104); an insulin syringe (101) in which insulin is pushed out according to the shift of the plunger gasket (102); and a housing having an opening for insulin outlet of mechanical insulin injection device.

Further, the portable mechanical insulin injection device of the present invention can use any type of insulin injection, such as meal-associated insulin injection or basal insulin injection. In case of meal-associated insulin injection, the insulin is injected before having a meal whereas the insulin is injected once a day in case of 24 hours effective basal insulin injection. Further, said insulin syringe in the portable mechanical insulin injection device of the present invention can be replaced by commercially marketed insulin cartridge.

The rotation of hand-operated rotating device (106) through gear rotating part (108) transfers its mechanically precise operation to the operating gear (105). Then, a plunger shifting screw (103) connected with operating gear (105) is rotated and the plunger shift lock nut (104) is moved for pushing and shifting the plunger gasket (102). Finally, insulin in the insulin syringe (101) is pushed out from the mechanical insulin injection device according to shift of plunger gasket.

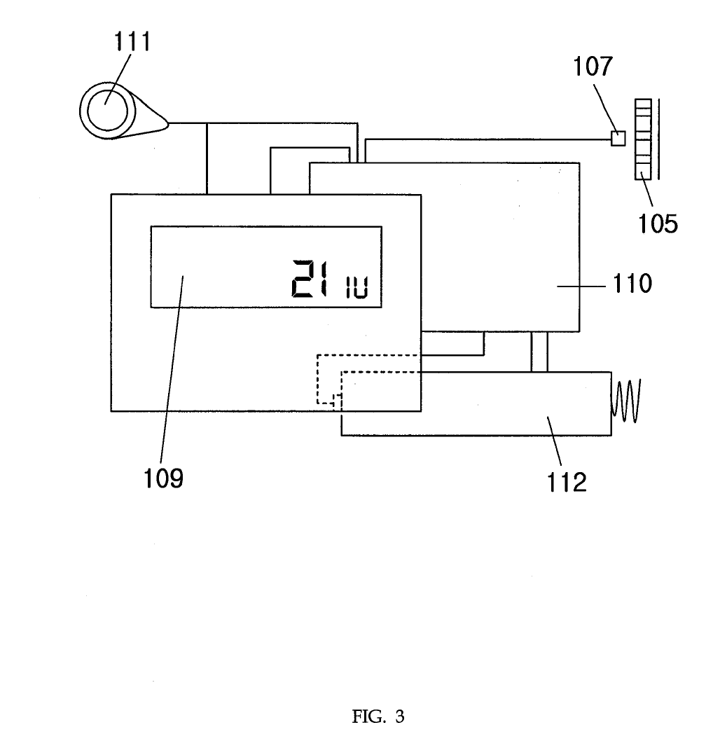

FIG. 3 shows a electrical part structure of the portable mechanical insulin injection device of the present invention.

In the mechanical insulin injection device (10) of the present invention, the injected insulin amount is displayed by liquid crystal display (109). Liquid crystal display (109) is connected with control panel (110) and pre-determined insulin amount is input to the control panel by the operation of control button (113). Sensor (107) connected with control panel (110) measures the injected insulin amount by detecting the rotation of gear. Then, the injected insulin amount is displayed by liquid crystal display (109) through the control panel (110).

When the pre-determined amount of insulin is almost injected, warning sound is generated from the warning sound speaker (111) according to the signal from control panel (110). This warning sound protects the user from being over injected according to hand-operation. Upon generating the warning sound, user slowly stops injecting the insulin by suspending the rotation of hand-operated rotation part (106).

For example, in case of injecting 30 IU of meal-associated insulin, the warning sound is generated at the time of about 25 IU of insulin. Then, the user slowly stop the rotation of hand-operated rotation part (106) in order to avoid over-injection of insulin.

Further, the remaining amount of insulin in the insulin syringe is also displayed by liquid crystal display (109) through control panel (110). This remaining amount is calculated by deducting the injected amount measured by sensor from initial insulin amount in the syringe.

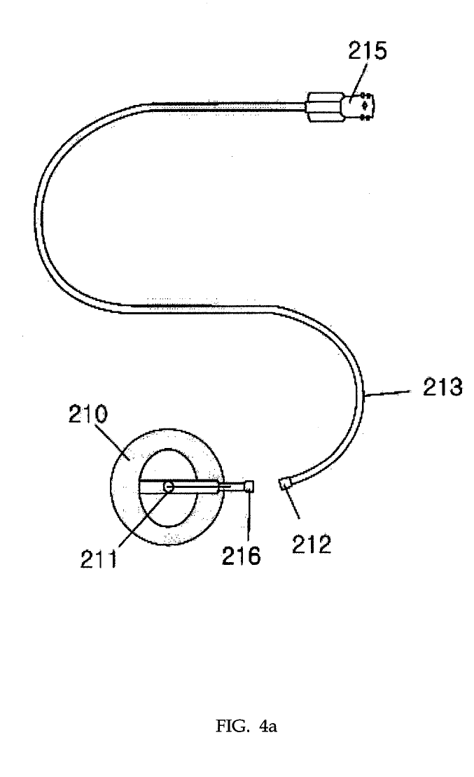

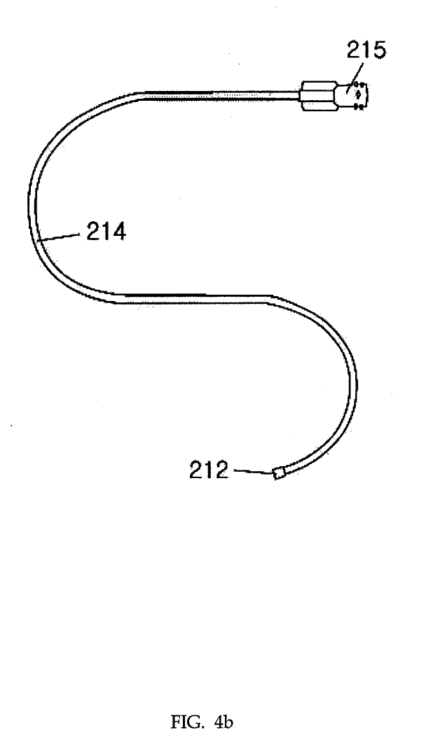

FIG. 4a shows a structure of insulin injection unit of the portable mechanical insulin injection device of the present invention.

As shown in FIG. 4a, an insulin injection needle (211) in the insulin injection unit (20) is inserted in the skin or muscle of patient. An insulin injection connector (215) is fixed with the insulin outlet of mechanical insulin injection device (10) at the time of insulin injection. Also, tubulate extension edge (212) and insulin injection edge part (216) shall be connected at the time of injecting insulin.

Further, meal-associated insulin and basal insulin can be alternatively injected through insulin injection unit (20), by replacing tubulate extension part (213).

FIG. 4b shows a replaceable part structure of insulin injection unit of the portable mechanical insulin injection device of the present invention.

As shown in FIG. 4b, a tubulate extension part in insulin injection unit (20) from insulin injection connector (215) to tubulate extension edge (212) is illustrated. Such tubulate extension part shall be replaced according to the change of insulin, such as, meal-associated insulin and basal insulin. Of course, insulin injection connector (215) shall be fixed with mechanical insulin injection device (10) for injecting insulin.

The advantageous effect of the present invention is to afford a portable mechanical insulin injection device comprising i) a mechanical insulin injection device (10) wherein said injection device is mechanically operated by hand-operated rotating part (106) whereas the amount of injected insulin and the pre-determined injection amount of insulin are electrically controlled and displayed by electrical control panel (110); and ii) an insulin injection unit (20) wherein an insulin injection needle (211) is inserted in the skin or muscle of patient and an insulin injection connector (215) is fixed with the insulin outlet of mechanical insulin injection device (10) at the time of insulin injection. Further, the tubulate extension part of insulin injection unit (20) can be replaced according to the kind of insulin, such as, meal-associated insulin and basal insulin.

What is claimed is:

1. A portable mechanical insulin injection device comprising
i) a mechanical insulin injection device (10) wherein said injection device is mechanically operated by hand-operated rotating part (106) whereas the amount of injected insulin and the pre-determined injection amount of insulin are electrically controlled and displayed by electrical control panel (110); and
ii) an insulin injection unit (20) wherein an insulin injection needle (211) is inserted in the skin or muscle of patient and an insulin injection connector (215) is fixed with the insulin outlet of mechanical insulin injection device (10) at the time of insulin injection,
wherein said mechanical insulin injection device (10) comprises
a) an operating gear (105) which is mechanically operated by the rotation of hand-operated rotating device (106) through gear rotating part (108);
b) a plunger shifting screw (103) connected with operating gear (105), so that said plunger shifting screw (103) shifts the plunger gasket (102) by moving the plunger shift lock nut (104);
c) an insulin syringe (101) in which insulin is pushed out according to the shift of the plunger gasket (102); and
d) a housing having an opening for insulin outlet of mechanical insulin injection device.

2. The portable mechanical insulin injection device according to claim 1, wherein said portable mechanical insulin injection device further comprises an electrical device comprising
i) a control panel (110) controlling the insulin injection amount upon inputting the pre-determined injection amount by control button (113) and measuring the injection amount by sensor (107);
ii) a liquid crystal display (109) controlled by control panel (110);
iii) a warning sound speaker (111) generated according to the around injection of pre-determined insulin amount; and
iv) battery (112) as electric power source.

3. The portable mechanical insulin injection device according to claim 1, wherein said insulin syringe (101) in the insulin injection device (10) is replaced by any type of insulin syringes such as meal-associated insulin syringe or with basal insulin syringe upon changing the tubulate extension part which is fixed at the insulin injection edge part (216).

4. The portable mechanical insulin injection device according to claim 1, wherein said insulin syringe (101) is replaced by commercially marketed insulin cartridge.
